(19) United States
(12) Patent Application Publication
(10) Pub. No.: US 2026/0204406 A1
(43) Pub. Date: Jul. 16, 2026

(54) METHOD, SYSTEM, AND COMPUTER PROGRAM ELEMENT FOR CONTROLLING AN INTERFACE DISPLAYING MEDICAL IMAGES

(71) Applicant: KONINKLIJKE PHILIPS N.V., EINDHOVEN (NL)

(72) Inventors: JOËL VALENTIN STADELMANN, MOSCOW (RU); ALEXANDER SELIVANOV, UNTERSCHLEIßHEIM (DE); HEINRICH SCHULZ, HAMBURG (DE)

(21) Appl. No.: 19/134,927

(22) PCT Filed: Nov. 30, 2023
(86) PCT No.: PCT/EP2023/083743

§ 371 Date: Jun. 2, 2025

(30) Foreign Application Priority Data
Dec. 9, 2022 (RU) 2022132290

(51) Int. Cl. G16H 40/63; G06F 3/01; G06V 10/25; G06V 40/18; G16H 10/60; G16H 30/20; G16H 40/20

(52) U.S. Cl. CPC G16H 40/63; G06F 3/013; G06V 10/25; G06V 40/18; G16H 10/60; G16H 30/20; G16H 40/20

(57) ABSTRACT

The invention concerns a method, a system and a computer program element for controlling an interface displaying medical images to a user on a display, comprising and configured to perform the steps of receiving a medical image of a patient, displaying the medical image on the display for the user to perform an image reading, identifying a region of interest in the medical image using an eye tracker configured to provide eye tracking data tracked from the user, analyzing the region of interest using a first artificial intelligence (AI) module, determining an anatomical structure from the medical image by correlating the identified region of interest with data from the first AI module, evaluating a status of the user using a second artificial intelligence (AI) module indicating an ability of the user to perform the image reading, controlling the interface and the displayed medical image depending on the eye tracking data.

### FIELD OF THE INVENTION

The invention relates to the field of controlling devices for a medical image reading of a user, and more specifically to a method for controlling an interface displaying medical images, a system for controlling an interface displaying medical images and a computer program element.

### BACKGROUND OF THE INVENTION

Image reading, like image reading of Chest X-rays (CXR) in a medical unit, for instance in an emergency department, is performed in a systematic manner, such that any time consuming can be avoided, and thus life-threatening improvisations and misreading of the medical image, and also to improve cost efficiency. The arrangement in which the image reading is performed is extremely codified, for example, which part of the medical image must be read.

The image readings are performed manually, which means the user, which usually is the radiologist, clicks with a computer mouse and manually navigates through the image and the study with this mouse. This interaction mode is time consuming. For example, the reading of a typical C×R image lasts on average 91 seconds. Further, there exist many distractions for radiologist when performing image reading, and an increasing workload, which may cause failure during image reading and image analysis.

### SUMMARY OF THE INVENTION

Therefore, there exists a need for optimizing and enhancing a reading process of a medical image, and a need for optimizing a controlling of an interface used for medical image reading. In particular, there exists a need for being able to control an interface used for displaying a medical image on a display during the image reading by a user. An analysis of the image reading should be improved and a new way of interacting with the interface, the workstation of the radiologist can be implemented, that is less cumbersome and therefore faster.

An object of the invention is to provide an improved method, a system, and a computer program element for controlling an interface displaying medical images.

The object of the present invention is solved with the subject matter of the independent claims, wherein further embodiments are incorporated in the dependent claims.

It should be noted that any feature, function and/or element described in the following with reference to the method equally applies to the system, and vice versa. Accordingly, any feature, function, step and/or element described in the following with reference to one aspect of the present disclosure equally applies to any other aspect of the present disclosure.

Further, it should be noted that some of the embodiments may be described with reference to chest X-ray images. Nevertheless, the embodiments are not limited to this application. The describes embodiments, described in the following with reference to one specific medical images equally applies to any other medical image, such as X-ray images, mammography images, MRI images, CT images may also be analyzed and the interface displaying this images could be controlled by the invention, wherein this list is only an example and not limiting.

According to a first aspect of the invention, a method for controlling an interface displaying medical images to a user on a display is described. The method comprises the steps of receiving a medical image of a patient, displaying the medical image at the display for the user to perform an image reading, Further the method comprises the steps of identifying a region of interest of the user in the medical image using an eye tracker configured to provide eye tracking data tracked from the user, analyzing the region of interest using a first artificial intelligence (AI) module. Then an anatomical structure is determined in a further step from the medical image by correlating the identified region of interest with data from the first AI module, evaluating a status of the user using a second artificial intelligence (AI) module indicating an ability of the user to perform the image reading, and controlling the interface and the displayed medical image depending on the eye tracking data.

In the context of the present invention, the term “image reading” shall be understood to describe the analysis of the medical image by a user, wherein the user is preferably a medical person, a doctor. The image reading is performed by the user by viewing at the display of the interface according to image reading guidelines, wherein these image reading guidelines represent medical standards for analyzing a medical image of a patient. These guidelines may vary depending on the country and the applied standard.

In the context of the present invention, the term “region of interest (ROI)” shall be understood to describe a sample within the medical image of particular interest for the medical diagnosis used by the user. For instance, a region of interest may be a boundary of a tumor, the bronchia, the lungs, the hearth, the bronchial tube, wherein this list is not limited. The kind and size of the ROI may depend on the medical image to be analyzed, the anatomical structure that could be found in the medical image, and a part of a body of the patient, which undergoes medical image analysis.

In the context of the present invention, the term “status of the user” shall be understood to describe, whether the user is in the physical and/or cognitive condition for performing a reading of a medical image. In particular, to status of the user shall indicate a possible inadequacy of the reading of the user, whether the user is tired or not. Hence, a fatigue status of the user may be monitored and/or analyzed.

In other words, a method is described which is used for controlling an interface which is configured for displaying one or more medical images to a user, wherein the user can perform a medical image reading of the displayed one or more medical images. The interface may be controlled by the user, which may be for instance a radiologist. In particular, the displaying of image information, part of the images, the display of the medical image and the display of the ROI may be controlled based on the eye tracking data. The monitored/tracked eye tracking data is used by the processor, may be stored in any suitable memory (local or online, cloud based and so on). The processor is configured to process the eye tracking data, for instance for providing this data to the respective AI modules used in the method and system. Moreover, the processor may configured for performing the steps of the method with respective electronic and computer elements. In particular, the method may be a computer implemented method, wherein the processor may be configured for performing at least some or all of the method steps as described in the embodiments. The advantage of the described method is that the user is able to control the interface without using any other devices, which means the user can control the interface without using a computer mouse or computer keys. Usually the interaction with the workstation, for instance the interface, happens through usual modalities, which are a mouse and the keyboard. These usual modalities may now be omitted, in particular may be replaced with the described method and system, therein the mouse and keys may be replaced by an eye tracker. One or more artificial intelligence modules may interpret the user's view, or the users gaze direction using the eye tracking in order to provide eye guided navigation. Further, a status of the user could be evaluated, in particular whether or not the user is able for performing a sufficient medical image reading. This means an artificial intelligence (AI) module may be used for helping the user to become aware of his own limitations during an image reading process. The method may evaluate the status of the user and indicate the ability of the user to perform the image reading, which means that the image reading may be evaluated when started and/or also during the image reading. Hence, the status of the user may be evaluated when starting a new image reading, or continuing an image reading, which has been stopped/paused. The eye tracker may be able to determine at any time the location in the medical image the user is currently looking at. By correlating this location with knowledge obtained from the first AI module, it is possible to infer the type of anatomical structure currently being investigated.

The controlling by an eye guided navigation depends on the respective eye tracking data, wherein each eye tracking data may be implemented in the method and system in a coded manner. For instance, the eye tracking data, which is detected, is how often the user is blinking and depending on the blinks, different actions may be carried out. Possible actions are for example, whether an image or a part of the image should be displayed, enlarged and/or removed from the display. The eye tracking data, which may be monitored by the eye tracker, will be elucidated in detail further below.

This method of interacting and method of controlling the interface may be less cumbersome and thus faster. The tracking of the user's eyes allows to control and monitor compliance with systematic review schemes. Further, when using the described method, the time taken to read each medical image may be reduced, which may result in a significant cumulative reduction of the resources allocated to medical image reading, which further may result in a sufficient reduction of exploitation costs.

According to an exemplary embodiment of the invention, the method may further comprise the step of simultaneously displaying on the display the current medical image and the region of interest, wherein the medical image and the region of interest may be displayed next to each other on the display. For instance, when examining a chest X-ray image, there are at least two images that can be displayed in such a manner that the images are arranged side by side, wherein on the left side the whole medical image is displayed and on the right side the ROI is displayed. It may also be possible that the images may be arranged above each other. Furthermore, the region of interest may be displayed in the medical image itself, wherein the ROI is displayed in the medical image in an indicated manner. For instance, the ROI is displayed/indicated with a marker and/or indicator in the medical image.

According to an exemplary embodiment of the invention, the step of displaying further comprises simultaneously displaying the current medical image of the patient and/or displaying a historical medical image of the patient on the display, wherein the first AI module analyses and indicates a region of interest of the historical medical image similar to the region of interest of the current medical image. According to this embodiment, the historical medical image and the current medical image may be displayed side by side or also above each other. Additionally, the ROI of the historical and/or of the current medical image may simultaneously displayed on the display of the interface. The ROI of the historical and/or of the current medical image may be indicated in the respective images itself and may further be displayed in an additional image on the display. The user may be able to control which image and which ROI should be displayed and in which manner they should be displayed, wherein the controlling may be elucidated in detail in further embodiments of the invention.

According to an exemplary embodiment of the invention, the method may further comprise the following steps of, depending on the received eye tracking data, displaying the region of interest by the first AI module in the medical image displayed on the display, generating an enhanced view of the indicated region of interest by the first AI module, and removing the enhanced view of the indicated region of interest by the first AI module. In other words, the user may be able to control the interface by using the eye tracker, whether a ROI should be generated and whether the ROI should be displayed or not and also how long the ROI should be displayed on the display of the interface. The enhanced view of the ROI may be displayed together with the current medical image at the display, wherein the medical image and the ROI may be displayed next to each other, side-by-side and/or above each other. An enhanced view may be understood to comprise (mean) an zoomed in or out view of the ROI, or rotated view of the ROI, which means that the ROI is displayed, for example, zoomed in and rotated image for allowing a better medical image reading.

According to an exemplary embodiment of the invention, wherein, when the eye tracker may determine that the user is staring at the region of interest for a period of time, indicating the region of interest with a boundary using the first AI module, wherein the boundary may be a rectangle, wherein the generating of the enhanced view may comprise improving the quality of this view, wherein, when the eye tracker determines that the user removes his view from the region of interest, removing the enhanced view of the region of interest.

Improving the quality of the enhanced view may comprise a contrast adaption, in particular a contrast enhancement using precomputed look-up tables, wherein for each respective organ to be examined a respective precomputed look-up table is provided. The contrast adaption may also use fly contrast adapting using either min-max normalization, histogram normalization or contrast limited adaptive histogram equalization. Further, the quality improvement may be a region-specific image filter, an edge enhancement, and/or high pass sharpening filters, and/or Canny's or Sobel's edge finding filter. The improving of the quality of the enhanced view may apply at least one of the above-mentioned features, on the other hand, a plurality of those features may be applied for improving the quality. Furthermore, the improved quality of the enhanced view of the ROI may comprise an improved and enlarged resolution of the medical image in the area of the ROI, or the enlarged resolution of the displayed ROI.

According to an exemplary embodiment of the invention, the step of analyzing of the region of interest using the first artificial intelligence (AI) module may comprise at least one of an object detection AI, a semantic segmentation AI, or an instance segmentation AI. Accordingly, it may also be possible that the analyzing of the ROI is performed with all of the AI modules, one after another or simultaneously, or only by on AI module, or by two AI modules. The object detection AI may use a deep convolutional neural network configured for object detection, wherein the object detection may be configured to find the object(s) in the image and may further be configured to indicate the object using for example a rectangle. The semantic segmentation AI may use a deep convolutional neural network configured for segmentation, which may mean that is assigns a corresponding and unique class label to each pixel in an image. For example, both lungs will be outputted (segmented) with the same color. The instance segmentation may combine both object detection and segmentation, in particular it may identify every item or instance of a class visible in an image and gives it a distinct mask or bounding box. For example, the instance segmentation may separate the lungs on right and left and an outputted color may be different. Furthermore, the object detection AI may use a Faster RCNN, or a YOLO algorithm. The semantic segmentation AI may use for instance DeepLab, UNet, or HRNet. The instance segmentation AI may use for instance Mask RCNN, MaskLab, TensorMask, YOLACT, SOLO or SOLOv2, or CenterMask. With the use of the object detection AI, it is possible to determine the anatomical structure at which the user is currently looking at. Further, with the information on which anatomical structure the user is focussing at, the system may be able to enhance ROI appropriate and provide appropriate guidance.

According to an exemplary embodiment of the invention, the method may further comprise the step of correlating the image reading of the medical image by the user with reading guidelines using the first AI module, and indicating at the medical image whether the image reading was performed in line with the reading guidelines or not. For instance, there may be several reasons for a user to pay more attention on a specific part of an image. For instance, for a chest X-ray image, the specific goal may be to find and analyze for a specific anomaly/disease, which may be located in the specific part of the image. However, incomplete reading of a medical image may/should be alerted and flagged, for instance with a visual marker displayed on the display of the interface. Alternatively, or additionally, the medical image, which was incompletely read, may be marked, which may be indicated, by the processor or by one of AI modules, that the reading was not performed according to the reading guidelines. Further, the incompletely read medical image may be added to a hanging queue such that a further reading and checking with the guidelines may be carried out later. Accordingly, the user may be authorized for making an incomplete reading, hence incomplete review of the image, and the incomplete review may only be indicated. For instance, in life-threatening situations, it is important that the user should be able to perform only a partial reading and able to complete a full formal reading later.

According to an exemplary embodiment of the invention, the step of controlling the interface may comprise at least one of controlling the size of the medical image, controlling a part of the medical image, controlling the size of the part of the medical image, controlling a scrolling through a plurality of medical images, controlling medical image data to be displayed, controlling a highlighting of the medical image and/or part of the medical image, controlling patient data displayable at the display with reference to the medical image. The controlling may be carried out for at least one of the above-mentioned features and/or for a plurality of these features. In particular, the step of controlling may comprise one single step of the above mentioned features or may comprise some and/or a plurality and/or all of them. Accordingly, the detected eye tracking data may be associated with a respective controlling feature. For instance, blinking once may confirm a displaying of an image, or an ROI. Another amount of blinking may be used for controlling the size of the ROI to be displayed, or the size of the enhanced view. A movement of the eye or the head may be used for controlling the scrolling through a plurality of images. The examples are not intended to be limiting, other eye tracking data may be used for other controlling features.

According to an exemplary embodiment of the invention, the eye tracking may be used to monitor eye tracking data which is at least one of an eye movement of the user, a viewing direction of the eyes of the user, an average fixation time of the eyes, a pupil area, a period of time at which the user has looked at a point on the display, an eye lid movement, an eye color, and a head movement of the user. For instance, an eye movement may be any movement of the eye which may be an up-and-down movement of the eye and/or and left and right movement of the eye. The monitored eye tracking data may be only be one of the above mentioned eye tracking data, or may be a plurality of these features or all features. It may be possible that one or more or all of the eye tracking data is tracked simultaneously. The eye tracking may be used to monitor the movement and behavior of each single eye of the user. The viewing direction of the eyes of the user may be a gaze determined from both eyes. An average fixation time of the eyes may be an average of any time at which the user is staring at a specific point in the medical image, wherein the duration of the average fixation time may be predefined by the respective reading standard. The pupil area and the change of this area may be measured for each single eye. Further, an eyelid movement may be an opening or closing of the eyelid, and/or one or more eyes blinking. The tracked eyes tracking data may be used for the second AI module to determine a fatigue of the user. For instance, the average fixation time and the pupil area may be used as features for fatigue detection using the fuzzy K-means clustering method. These features may be extracted from the pupil segmentation, either using computer vision method, such as thresholding, corrected elliptic Hough transform methods, or by deep learning methods, for instance using DeepLab architecture.

According to an exemplary embodiment of the invention, the first AI module may be trained with data from medical training images comprising information similar to the medical image to be displayed, wherein the medical training images used for training the first AI module may be annotated by medical specialist annotating anatomical structures in each of the medical training images. The trained data set may be an annotated data set, wherein this annotation is a per-pixel decision, as to which an object is present on this location or not. On the other hand, the first AI module may be a pre-trained model, which may be trained on a non-medical data.

According to an exemplary embodiment of the invention, the second AI module may be trained with a data set of iris images and pupil images obtained by the eye tracker, wherein the pupil is annotated on a per-pixel basis.

According to an exemplary embodiment of the invention, the eye tracker, the first AI module and the second AI module work simultaneously during the displaying of the medical image to the user on the display. Hence, the processor may be configured to perform all steps simultaneously, such that the image reading process can be made faster.

According to an exemplary embodiment of the invention, the second AI module may evaluate at least one of an eye movement, an eye lid movement, an eye color, or a pupil dilation for evaluating the ability of the user to perform the image reading. It may be possible that one, o a plurality of the above mentioned features is evaluated by the AI module, or also all of these features. Further, the evaluation of more than feature may be carried out simultaneously or one after another. In particular, the second AI module may evaluate a fatigue status as a status of the user, for determining the ability of the user to perform sufficient reading of the medical image. In particular, the fatigue may be evaluated by means of eye tracking. For instance, the eyes movements and pupil dilatation may be suitable markers for fatigue prediction. This is an advantage of notifying the user of the possible inadequacy of his or her reading. For instance, the monitoring of the eye color may monitor irritated eyes, or red eyes what might be used for indicating that the user is not able to look properly at the displayed image when detecting such irritated or red eyes.

According to a further exemplary embodiment of the invention the medical image to be displayed and which displaying should be controlled on the interface may be a chest X-ray image, an MRI image, a mammography image, an Ultra Sound image, a CT image, or a PET image.

According to a second aspect of the invention, a system is described for controlling an interface displaying medical images to a user. The system comprising a display comprising an interface for interfacing with a user. The system further comprise an eye tracker configured to provide eye tracking data tracked from the user. Further, the system comprises a processing unit configured for communicating with the display, wherein the processing unit is further configured to: receive a medical image of a patient and display the medical image at the display for the user for performing an image reading, identify a region of interest of the user in the medical image using the eye tracker, analyze the region of interest using a first artificial intelligence (AI) module, determine an anatomical structure from the medical image by correlating the identified region of interest with data from the first AI module, evaluate a status of the user using a second artificial intelligence (AI) module indicating an ability of the user to perform the image reading, and control the interface and the displayed medical images depending on the eye tracking data.

In other words, the system comprises a user interface, which may be controlled by the user using an eye tracker. In addition, the system may further comprise at least two AI modules, wherein at least one module analyzes specific part of the medical image currently looked at by the user in order to enhance the presentation of structures currently on the investigation and verify the user's adherence to systematic review guidelines. At least one of the modules may evaluate the user's fatigue and thus his or her ability to perform the image reading. The system uses the eye tracker for controlling, for instance, conventional viewer software, which may be implemented by at least two AI modules.

According to an exemplary embodiment of the invention, the system may further comprise a camera configured to communicate with the processor and configured for capturing at least one of an eye movement of the user, a viewing direction of the eyes of the user, an average fixation time of the eyes, a pupil area, a period of time at which the user has looked at a point on the display, an eye lid movement, or a head movement of the user. In particular, the camera may be the respective eye tracker, or may be used as a part of the eye tracker. The camera/the eye tracker may be placed near the display. The eye tracker allows to know at any time the location on the medical image where the user is currently focusing at, this can be achieved by using at least one of the above-described eye tracking features, which the camera may be configured to monitor. The camera, may be configured to capture at least one of the above mentioned features, or may capture all of them, or only a plurality of these features. In particular, the features captured by the camera may be captured simultaneously.

According to a third aspect of the invention, a computer program element for controlling an interface displaying medical images to a user is described. The computer program element, when being executed by a processor of a system is adapted to cause the system to receive a medical image of a patient and display the medical image at the display for the user for performing an image reading, identify a region of interest of the user in the medical image using an eye tracker configured to provide eye tracking data tracked from the user, analyze the region of interest using a first artificial intelligence (AI) module, determine an anatomical structure from the medical image by correlating the identified region of interest with data from the first AI module, evaluate a status of the user using a second artificial intelligence (AI) module indicating an ability of the user to perform the image reading, control the interface and the displayed medical images depending on the eye tracking data.

The computer program element may be part of a computer program, but it can also be an entire program by itself. For example, the computer program element may be used to update an already existing computer program to get to the present invention.

The program element may be stored on a computer readable medium. The computer readable medium may be seen as a storage medium, such as for example, a USB stick, a CD, a DVD, a data storage device, a hard disk, or any other medium on which a program element as described above can be stored.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

It has to be noted that embodiments of the invention have been described with reference to different subject matters. In particular, some embodiments have been described with reference to apparatus/system type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered as to be disclosed with this application.

### BRIEF DESCRIPTION OF THE DRAWINGS

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

FIG. 1 illustrates a flow diagram of a method according to an embodiment of the invention.

FIG. 2 illustrates a medical image displayed on an interface according to an embodiment of the invention.

FIG. 3 illustrates a further medical image displayed on an interface according to an embodiment of the invention.

FIG. 4 illustrates a further medical image displayed on an interface according to an embodiment of the invention.

### DETAILED DESCRIPTION OF EMBODIMENTS

The illustrations in the drawings are schematic. It is noted that in different figures similar or identical elements are provided with the same reference signs.

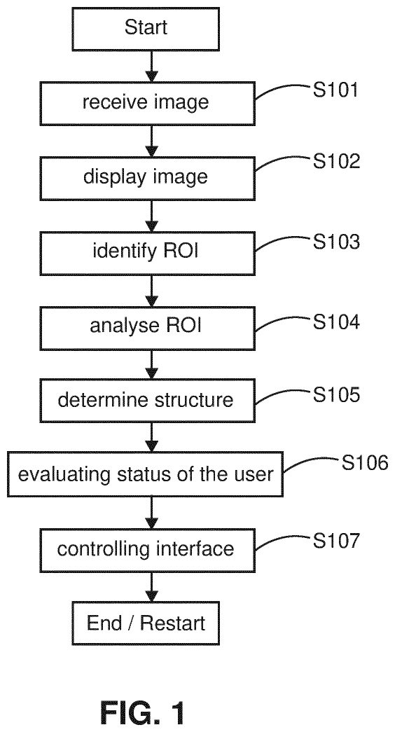

FIG. 1 illustrates a flow diagram comprising method steps according to an embodiment of the invention. The method for controlling an interface displaying medical images to a user on a display is described with the following steps. In step 101, a medical image of a patient is received and in step 102, the medical image is displayed at the display for the user such that the user can perform an image reading. In step 103, a region of interest of the user in the medical image is identified using an eye tracker, which is configured to provide eye tracking data tracked from the user. When the region of interest is identified then in step 104 the region of interest is analyzed using a first artificial intelligence (AI) module. In 105, an anatomical structure from the medical image is determined by correlating the identified region of interest with data from the first AI module. In step 106 a status of the user is evaluated using a second artificial intelligence (AI) module indicating an ability of the user to perform the image reading. In step 107 the interface and the displayed medical image is controlled depending on the eye tracking data. The steps of the method may be performed one after another wherein the order as presented herein is not limiting, because the order of the steps may be different. Further, some or at least a plurality of the method steps may be performed simultaneously. For instance, the evaluation of the status of the user may be performed simultaneously during the whole image reading by the user. Accordingly, the evaluation of the status of the user may be performed simultaneously with the identification of the ROI and the analysis of the ROI and the determination of the anatomical structure. Furthermore, the method may further comprise additional sub steps, for instance after evaluating the status of the user may be indicated and the indication may be displayed in another method step on the display for the user. It may also be possible that the method is restarted after controlling the interface because the image reading is not complete and the user want to continue with the image reading, when the image reading was flagged as incomplete. Hence, the steps may start after step 107 again, for instance, by displaying the image in step 102 when continuing with the image reading the same image may be displayed again. On the other hand, it may be possible that a new image is received from the same patient and a further image reading process should be performed for this image. The step of displaying the medical image may comprise the following further sub steps, displaying the region of interest using the first AI module in the medical image, and generating an enhanced view of the indicated region of interest by the first AI module, wherein the displaying of the ROI, the displaying of the enhanced view and also displaying of the medical image itself is controlled depending on the eye tracking data.

In particular, the step of controlling the interface may comprise further sub steps of controlling the size of the medical image, controlling a part of the medical image, controlling the size of the part of the medical image, controlling a scrolling through a plurality of medical images, controlling medical image data to be displayed, controlling a highlighting of the medical image and/or part of the medical image, controlling patient data displayable at the display with reference to the medical image. Each of the sub steps may be controlled by the user depending on the eye tracking data.

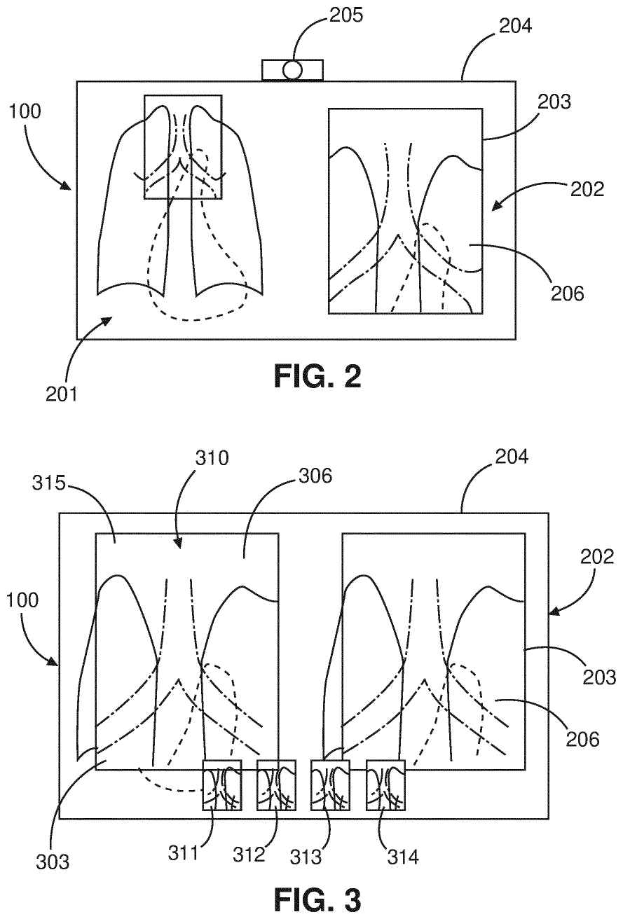

FIG. 2 shows a system for controlling an interface 100 displaying medical images 201 to a user according to one embodiment of the invention. The system comprising a display 204 comprising an interface 100 for interfacing with a user. The system further comprises an eye tracker 205 configured to provide eye tracking data tracked from the user. Further, the system comprises a processing unit configured for communicating with the display 204. The processing unit is configured to receive the medical image 201 of a patient and display the medical image 201 at the display 204 for the user for performing an image reading. Further, the processing unit is configured to identify a region of interest of the user in the medical image 201 using the eye tracker 205. The region of interest in FIG. 2 is indicated with a rectangle on the left side of the display 204. The region of interest is analyzed using a first artificial intelligence (AI) module. An anatomical structure 206 is determined from the medical image 201 by correlating the identified region of interest with data from the first AI module. A status of the user is evaluated by the processor using a second artificial intelligence (AI) module, indicating an ability of the user to perform the image reading. The interface 100 and the displayed medical images 201 is/are controlled depending on the tracked eye tracking data. On the display 204, the current medical image 201 and the region of interest (rectangle) are simultaneously displayed. In FIG. 2 the medical image 201 and the enhanced view 202 of the region of interest are displayed next to each other on the display 204. On the left side the current medical image is shown 201 with the indicated ROI and on the left side of the display 204 the enhanced view 202 is shown. If the user stares at a specific structure of the medical image 201 the interface highlights the boundaries 203 as the ROI with a rectangle line. The boundaries 203 are known from the first AI module, which uses an object detection, and/or semantic segmentation for determining the anatomical structure 206 at which the user is staring. For instance, the enhanced view 202 is a zoomed in version of the ROI with adjusted contrast for optimizing and easing the image reading by the user. The controlling of the detection of the ROI by the AI module may be confirmed by the user with a blink. For instance, the identified ROI is marked with the rectangle 203 in the medical image 201 and the user confirms this identification by a single blink, for a simple blinking code for “yes/correct”. If the ROI is not the corrected ROI the user may use another blinking code like blinking two times for “no” or the eye tracker may analyses the head movement, such that for example the shaking of the head of the user from a left to the right side, or vice versa, may be understood as a “no”. If the user is satisfied with his investigation and/or has finished the reading of the enhanced view 202, the user can let his or her gaze return to the overview 201, i.e. the whole medical image 201 on the left side of the display 204 and the enhanced view 202 and/or the highlighted ROI 203 is removed from the display 204. In FIG. 2, the displayed medical image 201 is the image of a chest of a patient and the identified ROI indicated with the boundaries 203 are the airways. The enhanced view 202 with improved image quality (the quality of the enhanced view 202 may be improved using the features as described with the embodiments in the sections above) is the airways, in particular the bronchioles.

The eye tracker 205 is placed in close proximity to the user's screen, which is here on top of the display 204. The eye tracker is a camera 205 configured to communicate with the processor and configured for capturing at least one or more of an eye movement of the user, a viewing direction of the eyes of the user, an average fixation time of the eyes, a pupil area, a period of time at which the user has looked at a point on the display, and a head movement of the user. Accordingly, the eye tracker 205 knows all times the location on the medical image 201 the user is currently looking at.

FIG. 3 shows the displaying of another medical image 3 according to an embodiment of the invention. In FIG. 3 a historic medical image is displayed, wherein a plurality of historical images 311-314 are displayed in a row at the bottom of the display 204. On the left side of the display, the ROI of the historic medical image 310 is displayed and on the right side of the display 204 the current ROI of the current medical image is displayed. Accordingly, the step of displaying comprises simultaneously displaying the current medical image 201 of the patient and/or displaying a historical medical image of the patient on the display 204, wherein the first AI module analyses and indicates a region of interest 303 of the historical medical image similar to the region of interest 203 of the current medical image 201. The indication of the region of interest, for example the boundary 303, 203, may comprise a color coding, such that a confusing regarding the nature of the image: current or historic can be avoided. For example, the boundary of the region of interest 303 of the historic image is colored with a different color than the boundary 203 of the current medical image 201.

The interpretation of eye tracking data allows several possibilities. For instance, blinking three times may be defined as a signal to bring on screen of patient's history. The interpretation of the eye tracking data may also be specified by the user, which means the user can change settings according to his or her preferences. In FIG. 3 a blinking for three times brings up a historic images 311-314 of a patient, which are displayed on the lower row and one specific historic image is highlighted on the left. The chosen historic medical image is confirmed by the user by a blinking of the eye, or a nodding of the head. For instance, a scrolling through the historic images 311-314 in the row may be controlled by moving the head to the left side for a picture on the left side of the row or by moving the head to the right side for a picture on the right side of the row. The eye tracker 205 is tracking these head movements, the eye tracking data is analyzed by the processor and the interface is controlled for what is to be displayed. Moreover, on the medical image and/or on the ROI 303 respective image data 315 could be displayed, for example this information can be displayed in the upper left corner of the ROI 303. The displayed information may comprise a date when the image was taken (for an historic medical image), or any other relevant patient data, like age, disease etc.

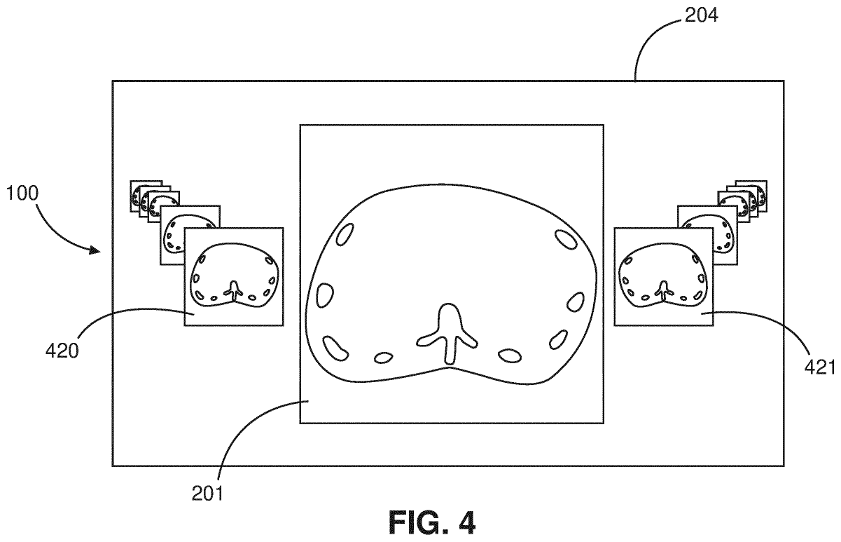

FIG. 4 shows a further medical image displayed on an interface according to an embodiment of the invention. The invention may also be applied to 3-D medical images, which is illustrated in FIG. 4. During a generation of CT, MRI, or PET images, a body part of a patient is subdivided into a set of slices. A single image may be such slice and for analyzing the full body part, each image slice must be read by the user. As illustrated in FIG. 4 each slice can be displayed on the interface 100 as a single medical image 201. The user is able to control the interface 100 for instance using a head movement for scrolling through each single slide by a moving the head to the left for choosing a slide 420 before the present image 201 and/or by moving to head to the right for choosing a slide 421 after the present image 201. It may also be possible that the interface 100 is controlled by an eye movement similar to the left or to the right for scrolling through the different medical image slides.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. It should be noted that the term “comprising” does not exclude other elements or steps and “a” or “an” does not exclude a plurality. Also, elements described in association with different embodiments may be combined. It should also be noted that reference signs in the claims should not be construed as limiting the scope of the claims

LIST OF REFERENCE SIGNS:

| | |
|---|---|
| 100 | interface |
| 201 | medical image |
| 202 | enhanced view |
| 203, 303 | boundary |
| 204 | display |
| 205 | eye tracker |
| 206, 306 | anatomical structure |
| 310 | historical image |
| 311-314 | historical images |
| 315 | image data |
| 420 | image slice |
| 421 | image slice |
| S101-S107 | steps |

1. A method for controlling an interface displaying medical images to a user on a display, the method comprising:
receiving a medical image of a patient;
displaying the medical image at the display for the user to perform an image reading;
identifying a region of interest of the user in the medical image using an eye tracker configured to provide eye tracking data tracked from the user;
analyzing the region of interest using a first artificial intelligence (AI) module;
determining an anatomical structure from the medical image by correlating the identified region of interest with data from the first AI module;
evaluating a fatigue status of the user using a second artificial intelligence (AI) module indicating an ability of the user to perform the image reading, wherein the second AI module uses the eye tracking data from the eye tracker; and
controlling the interface and the displayed medical image depending on the eye tracking data.

2. The method according to claim 1, further comprising simultaneously displaying on the display the current medical image and the region of interest, wherein the medical image and the region of interest are displayed next to each other on the display.

3. The method according to claim 1, further comprising simultaneously displaying the current medical image of the patient and/or displaying a historical medical image of the patient on the display, wherein the first AI module analyses and indicates a region of interest of the historical medical image similar to the region of interest of the current medical image.

4. The method according to claim 1 further comprising:
displaying the region of interest by the first AI module in the medical image displayed on the display;
generating an enhanced view of the indicated region of interest by the first AI module;
removing the enhanced view of the indicated region of interest by the first AI module.

5. The method according to claim 4, wherein, when the eye tracker determines that the user is staring at the region of interest for a period of time, indicating the region of interest with a boundary using the first AI module, wherein the boundary is a rectangle, wherein the generating of the enhanced view comprises improving the quality of the enhanced view of the medical image, wherein, when the eye tracker determines that the user removes his view from the region of interest, removing the enhanced view of the region of interest.

6. The method according to claim 1, further comprising:
wherein analyzing the region of interest using the first AI module comprises at least one of an object detection AI, a segmentation AI, and an instance segmentation AI,
wherein the object detection AI uses a deep convolutional neural network configured for object detection,
wherein the segmentation AI uses a deep convolutional neural network configured for segmentation.

7. The method according to claim 1, further comprising
correlating the image reading of the medical image by the user with reading guidelines using the first AI module,
indicating at the medical image whether the image reading was performed in line with the reading guidelines or not.

8. The method according to claim 1, wherein controlling the interface comprises at least one of controlling the size of the medical image, controlling a part of the medical image, controlling the size of the part of the medical image, controlling a scrolling through a plurality of medical images, controlling medical image data to be displayed, controlling a highlighting of the medical image and/or part of the medical image, and controlling patient data displayable at the display with reference to the medical image.

9. The method according to claim 1, wherein the eye tracking is used to monitor eye tracking data which is at least one of an eye movement of the user, a viewing direction of the eyes of the user, an average fixation time of the eyes, a pupil area, a period of time at which the user has looked at a point on the display, an eye lid movement, an eye color, and a head movement of the user.

10. The method according to claim 1,
wherein the first AI module is trained with data from medical training images comprising information similar to the medical image to be displayed,
wherein the medical training images used for training the first AI module are annotated by medical specialist annotating anatomical structures in each of the medical training images, and/or
wherein the first AI module is a pre-trained AI module trained on non-medical data.

11. The method according to claim 1, wherein the eye tracker, the first AI module and the second AI module work simultaneously during the displaying of the medical image to the user on the display.

12. The method according to claim 1, wherein the second AI module evaluates at least one of an eye movement, eye lid movement, an eye color, and a pupil dilation for evaluating the ability of the user to perform the image reading.

13. A system for controlling an interface displaying medical images to a user, the system comprising:
a display comprising an interface for interfacing with a user;
an eye tracker configured to provide eye tracking data tracked from the user;
a processor configured to communicate with the display, wherein the processor is further configured to:
receive a medical image of a patient;
display the medical image at the display for the user for performing an image reading;
identify a region of interest of the user in the medical image using the eye tracker;
analyze the region of interest using a first artificial intelligence (AI) module;
determine an anatomical structure from the medical image by correlating the identified region of interest with data from the first AI module;
evaluate a fatigue status of the user using a second artificial intelligence (AI) module indicating an ability of the user to perform the image reading, wherein the second AI module uses the eye tracking data from the eye tracker; and
control the interface and the displayed medical images depending on the eye tracking data.

14. The system according to claim 12, further comprising a camera configured to communicate with the processor and configured for capturing at least one of an eye movement of the user, an eye lid movement, a viewing direction of the eyes of the user, an average fixation time of the eyes, a pupil area, a period of time at which the user has looked at a point on the display, and a head movement of the user.

15. (canceled)
